(19) United States
(12) Patent Application Publication
(10) Pub. No.: US 2021/0106218 A1
(43) Pub. Date: Apr. 15, 2021

(54) VISUAL FIELD TEST DEVICE

(71) Applicant: Vision for Mars Technologies LLC, South Jordan, UT (US)

(72) Inventors: Neil Griffin, Melbourn (GB); Roger Clarke, Melbourn (GB)

(73) Assignee: Vision for Mars Technologies LLC, South Jordan, UT (US)

(21) Appl. No.: 16/941,589

(22) Filed: Jul. 29, 2020

(30) Foreign Application Priority Data
Oct. 11, 2019 (GB) 1914761.0

(51) Int. Cl. A61B 3/024; G09G 3/34; G06F 3/14; G09G 3/36; A61B 3/00; A61B 3/14; A61B 3/113

(52) U.S. Cl. CPC A61B 3/024; G09G 3/3406; G06F 3/1423; G09G 3/3648; G09G 3/3607; G09G 2354/00; A61B 3/14; A61B 3/0033; A61B 3/113; A61B 3/0091; A61B 3/005

(57) ABSTRACT

A visual field device includes a light source and an eyepiece. The light source includes a backlight arranged to generate, in use, light directed towards a first liquid crystal display (LCD) screen and a second LCD screen, the first LCD screen and the second LCD screen overlapping one another and the backlight such that the light source outputs light transmitted through both the first and second LCD screens. The eyepiece is arranged between the light source and a subject position, the eyepiece being configured to receive light output by the light source and focus the received light towards the subject position. Each of the first and second LCD screens include a respective array of pixels, each pixel being controllable so as to vary its transmittance to light generated by the back-light.

### GOVERNMENT SUPPORT

This invention was made with government support under Federal Award No. NNX16A069A awarded by the National Aeronautics and Space Administration. The government has certain rights in the invention

### CROSS-REFERENCE TO RELATED APPLICATIONS

The present application claims priority to GB Patent Application No. 1914761.0, entitled “Visual Field Test Device,” filed Oct. 11, 2019, the entirety of which is incorporated by reference herein.

### FIELD

The present disclosure relates to visual field test devices and methods of performing visual field tests using the same.

### BACKGROUND

Visual field tests may be used to analyze the visual field of a patient, i.e. the area that is visually perceptible to the patient while the patient's vision is focused on a central point. Visual field tests find application in the assessment and diagnosis of various ophthalmic defects and diseases and other medical conditions, for example glaucoma, macular degeneration, refractive errors, cataracts, stroke, pituitary disease and neurological conditions, which may impair or otherwise alter the visual field of the patient.

The term “perimetry” refers to visual field tests in which the patient is presented with stimuli, typically against a uniform background, at various positions in his visual field while his gaze is focused on a fixed target. The patient indicates whether he can see the stimuli at each position, for example by communicating with a technician supervising the test or by the use of some input device such as a button, and this information is used to measure the extent of the visual field. As well as being capable of establishing the extent and shape of the outermost perimeter of the visual field, perimetry tests are capable of identifying defects across the field of vision such as scotomata (which may be symptoms of glaucoma, strokes or injury, for example). The background may be provided by, for example, a uniformly illuminated bowl-shaped cavity, and the stimuli as bright spots against this background (for example as lights arranged within the cavity or by projecting spots of light onto the background). The stimuli are typically presented one at a time, and may either be static (i.e. each at a respective fixed position against the background) or moving (for example moving towards the fixed target from a starting position outside of the patient's field of view).

Perimetry relies on the patient's reported perception of the stimuli presented to him being accurate, and the stimuli presented must be controllable over a wide range of luminances. For this reason, conventional perimetry devices employ light sources such as projectors that are capable of producing stimuli with a wide dynamic range, typically on the order of 10,000:1 between the luminance of the brightest stimulus (or stimuli) and that of the dimmest stimulus presented to the patient. However, perimetry devices of this kind are generally large in size owing to the fact that the screen across which the stimuli are presented must extend across a sufficient area to allow the periphery of the patient's field of view to be studied. This makes conventional perimetry devices impractical to transport and limits the nature of the environments in which they may be deployed to those to which the devices can be transported and which have sufficient space to accommodate them. There is hence a need for more compact visual field test devices.

### SUMMARY

A first aspect of the present disclosure provides a visual field test device comprising:
a light source comprising a backlight arranged to generate, in use, light directed towards a first liquid crystal display (LCD) screen and a second LCD screen, the first LCD screen and the second LCD screen overlapping one another and the backlight such that the light source outputs light transmitted through both the first and second LCD screens; and
an eyepiece arranged between the light source and a subject position, the eyepiece being configured to receive light output by the light source and focus the received light towards the subject position;
wherein each of the first and second LCD screens comprises a respective array of pixels, each pixel being controllable so as to vary, in use, its transmittance to light generated by the back-light such that changing the transmittance of one or more pixels at corresponding positions in each of the first and second LCD screens relative to the surrounding pixels produces a stimulus perceptible when viewed through the eyepiece from the subject position.

The term “liquid crystal display screen” (“LCD screen”) as used herein refers to a screen comprising at least a layer of liquid crystal controllable so as to vary the transmittance of the LCD screen to radiation. For example, an LCD screen could include a layer of liquid crystal which responds to the application of a voltage by the alignment of its molecules along one or more axes. Each LCD screen may include two polarizing filters, one disposed on either side of the layer of liquid crystal. The axes of transmission of the two polarizing filters may be substantially perpendicular to one another such that if the liquid crystal layer between them is not set to alter the polarization of radiation passing through it, the transmittance of the screen is substantially zero (since radiation transmitted through the screen encounters two orthogonal polarizing filters, one after the other, without experiencing any additional polarization between the two). Detailed examples of LCD screens will be described below. It should be noted that “changing” the transmittance of one or more pixels as defined above may include increasing or decreasing the transmittance.

Combining two LCD screens in the manner described above yields a light source capable of producing visual stimuli of a sufficiently high dynamic range to perform visual field tests. While typical LCD screens are capable of producing visual features with a dynamic range of less than 1,000:1, a light source that outputs light transmitted through two such screens enables a far higher contrast to be achieved. For example, if the transmittance of each screen in one region of the light source is set to 1 per cent, then the transmittance of the two screens in combination in that region will be around 0.01 per cent. If the transmittance of each screen in an adjacent region of the light source is then set to a value close to 50 per cent, the contrast ratio between the adjacent regions will be on the order of 2,500:1 (since the fraction of light transmitted through the more opaque region would be around 0.04 per cent of that transmitted in the adjacent region). Hence, at least one, or both, of the first and second LCD screens is configured to be controlled to display features having a contrast ratio of at least 1,000:1. This may allow stimuli to be presented to the patient at the subject position having a luminance that contrasts with that of a background displayed by the light source at a ratio of up to approximately 65,000:1. It should be noted that the term “luminance” as used throughout this specification has its usual meaning and refers to the luminous intensity (i.e. power weighted by wavelength based on the sensitivity of the eye) per unit area travelling in a given direction (in the context of visual field tests, the relevant direction will usually be a direction leading towards the eye of the patient), and is typically reported in units of candela per square meter (cd/m²), also referred to as “nits”.

Further to the above, the combination of a light source and eyepiece as defined above may provide a more compact visual field test device. In particular, the eyepiece may be configured to focus the received light towards the subject position across range of angles (with respect to the line extending from the subject position to the eyepiece) having a magnitude greater than the angle that would be subtended by the light source in the field of view of an observer positioned at the subject position without the eyepiece being present, thus causing the stimuli produced by the light source to be presented across a greater range of angles in the field of view of a patient at the subject position that would be achieved if the light source were at the same separation from the subject position without the eyepiece. Hence, in some embodiments the eyepiece is configured to focus the received light towards the subject position across a range of angles having a magnitude greater than the angle subtended by the light source when viewed from a distance equal to the distance between the subject position and the light source.

In embodiments the pixels of the first LCD screen may be colorless. This means that the pixels of the first LCD screen do not impede certain visible wavelengths significantly more than others when transmitted therethrough, and can thus be controlled to affect the opacity of the first LCD screen without significantly affecting the color of the transmitted light. This enables the first LCD screen to be controlled so as to vary the brightness of the light source across a ‘greyscale’ range of values. This may also avoid the need to position the two LCD screens very precisely to keep like color pixels in alignment, since two differently colored pixels overlapping one another may in combination be substantially or completely opaque (because of the resulting combination of two differently-colored filters).

In yet further embodiments, the array of pixels of the second LCD screen comprises pixels each having one of a plurality of colors, the plurality of colors may include red, green and blue. By selectively controlling differently-colored pixels, the second LCD screen may be made to present stimuli of different colors to the patient. It should be understood that in such embodiments the second LCD screen may be arranged on either side of the first LCD screen, i.e. the light from the backlight could be transmitted through the colored pixels of the second LCD screen before or after being transmitted through the first LCD screen. In some embodiments, the pixels of one of the first LCD screen are colorless and the pixels of the second LCD screen comprises pixels each having one of a plurality of colors as described above. Again, in these embodiments either the first or second LCD screen could be arranged nearest the backlight.

In some embodiments the visual field test device further comprises a focusing target positioned between the back-light and the subject position such that a patient at the subject position is able to focus his gaze on the focusing target. The focusing target may be disposed on the light source (e.g. as a painted or printed feature or as a physical element fixed to the light source) or be separate from it and positioned between the light source and the subject position. A focusing target may alternatively be provided by controlling the first and second LCD screens so as to present an image, pattern or other feature on the light source suitable for focusing the vision of the patient.

In some embodiments, the visual field test device further comprises a rest adapted to support, in use, the head of a patient at the subject position. The rest enables the patient to stabilize the position of the eye at the subject position, which can improve the reliability of a visual field test performed using the device by ensuring that the positions at which the stimuli are presented correspond to the intended positions in the patient's field of view.

In some embodiments, the visual field test device further comprises a feedback device configured to receive feedback from a patient at the subject position during a visual field test in which one or more stimuli perceptible at the subject positon are produced by the light source. The feedback device could be, for example, a button that the patient may be instructed to press each time he perceives a stimulus during the visual field test. The feedback device may be configured to output data to a processor configured to produce a record of the feedback received from the patient, which may be compared (possibly by the processor) to the actual sequence of stimuli presented in order to assess the patient's visual field.

In some embodiments the visual field test device further comprises a camera configured to monitor the position of a pupil of the eye of a patient at the subject position. The position and orientation of the patient's pupil are determined by the direction in which his vision is focused, so the information yielded by the camera may be used to confirm that the patient's vision is focused on the correct position (typically the center of the light source) at all times during a visual field test. The visual field test device may also include an illuminating source configured to illuminate the subject position with radiation detectable by the camera such that such that the camera may monitor the position of the pupil of the eye by recording the radiation reflected by the eye. Illuminating the eye may ensure that the camera receives radiation from it sufficient to monitor its position. However, if the eye is illuminated with visible light, this may distract the patient and possibly reduce the quality of the visual field test. Hence, in some embodiments, the radiation is not in the visible portion of the electromagnetic spectrum, and may include one or more infra-red wavelengths. As the patient will not be able to perceive wavelengths outside of the visible part of the spectrum, the illuminating source will not distract him in these embodiments.

In yet further embodiments, the visual field test device further comprises an optical component arranged to direct the radiation produced by the illuminating source towards the eye and/or direct the radiation reflected by the eye towards the camera. This allows the camera and the illuminating source to be arranged in a more compact manner than if the camera and the illuminating source were arranged so as to each direct light to or receive light from the subject position along a direct line of sight. It should be noted that the radiation from the illuminating source may or may not be directed through the eyepiece. In some embodiments, however, the illuminating source is configured to direct the radiation towards the eyepiece, whereby the radiation is directed towards the subject position. This allows the illuminating radiation to be directed towards the eye along the direction that it is intended to face during the visual field test (i.e. the direction between the subject position and the eyepiece). In some embodiments, the optical component comprises a partial mirror arranged between the light source and the eyepiece, the partial mirror being configured to: permit light from the light source to be transmitted therethrough towards the eyepiece, and to reflect the radiation produced by the illuminating source towards the eyepiece, whereby the radiation is directed towards the subject position, and/or reflect the radiation reflected by the eye through the eyepiece towards the camera. This provides a particularly compact configuration of the light source, eyepiece, camera and illuminating source.

A second aspect of the present disclosure provides a method of performing a visual field test, the method comprising:
controlling a backlight to generate light directed towards a first LCD screen and a second LCD screen, the first LCD screen and the second LCD screen overlapping one another and the backlight such that the light source outputs light transmitted through both the first and second LCD screens, wherein each of the first and second LCD screens comprises a respective array of pixels, each pixel being controllable so as to vary, in use, its transmittance to light from the back-light, and wherein an eyepiece is arranged between the light source and a subject position, the eyepiece being configured to receive light output by the light source and focus the received light towards the subject position; and
increasing the transmittance of one or more pixels at corresponding positions in each LCD screen relative to the surrounding pixels so as to produce one or more stimuli perceptible when viewed through the eyepiece from the subject position.

This method provides all of the advantages achieved by the visual field test device of the first aspect, and may be performed using a visual field test device as described herein.

The stimuli may be small, relatively bright or dark features on the light source, and may be defined by, for example, a group of pixels in each of the first and second LCD screens contained within a region that constitutes no more than a predetermined fraction (e.g. 1 per cent) of the total area of the light source. In some embodiments, the pixels of each of the first and second LCD screens outside of a first region are controlled so as to produce a uniform background which surrounds the first region. The luminance of the background as seen from the subject position may be less than or greater than that of the first region, depending on whether the stimulus presented is a bright or dark feature. By providing a background that is uniform (i.e. with substantially the same brightness across its whole extent), the likelihood of variations in the brightness of the background being mistaken for stimuli by the patient is reduced.

In some embodiments, the first and second LCD screens are controlled such that the contrast ratio between the luminance of the first region and that of the uniform background as perceived from the subject position is at least 65,000:1. As explained above, this allows the sensitivity of the visual response to be tested over a wide range of luminance.

In some embodiments, the method further comprises controlling the first and second LCD screens to as to produce a focusing target perceptible from the subject position and suitable for focusing the vision of a patient at the subject position. A focusing target enables the patient to focus his vision on the intended position, which will preferably be the center of the light source. In other embodiments, a focusing target could be provided by a feature disposed between the light source and the subject position (e.g. on the light source) as described above.

In some embodiments the visual field test further comprises presenting a plurality of stimuli each at a respective position on the light source, each respective position corresponding to a position in the field of view of a patient at the subject position. It should be understood that the stimuli may be presented one-by-one, or more than one at a time. In some embodiments, the plurality of stimuli are presented in accordance with a predetermined routine. The predetermined routine may define, for example, one or more of the position of stimulus, the time for which it is presented, the order in which the stimuli are presented and the color of each stimulus, and the color of each stimulus, if the first and/or LCD screens comprise colored pixels as described above with reference to the first aspect.

### LIST OF FIGURES

Examples of a visual field test devices and visual field tests will now be described with reference to the accompanying drawings, in which:

FIG. 1A depicts a schematic representation of a conventional visual field test device;

FIG. 1B schematically depicts a screen of the visual field test device of FIG. 1A as seen from a subject position;

FIG. 2A schematically depicts a visual field test device, according to one or more embodiments shown and described herein;

FIG. 2B schematically depicts an enlarged portion of part of FIG. 2A, according to one or more embodiments shown and described herein;

FIG. 3A schematically illustrates a cross-sectional view of a light source of a visual field test device such as illustrated in FIGS. 2A and 2B, according to one or more embodiments shown and described herein;

FIG. 3B schematically illustrates a cross-sectional view of a light source of a visual field test device such as illustrated in FIGS. 2A and 2B, according to one or more

embodiments shown and described herein,

FIG. 4A illustrates a face-on view of a light source such as illustrated in FIGS. 3A and 3B, according to one or more embodiments shown and described herein;

FIG. 4B schematically illustrates an enlarged view of a region of the light source of FIG. 4A, according to one or more embodiments shown and described herein and

FIG. 5 depicts a flow chart for a visual field test, according to one or more embodiments shown and described herein.

### DETAILED DESCRIPTION

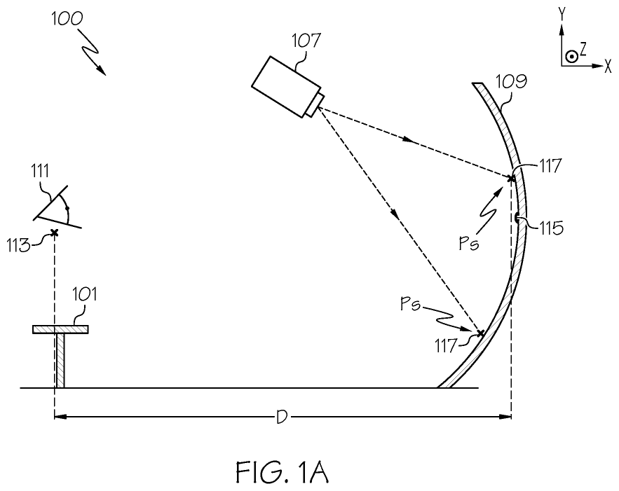

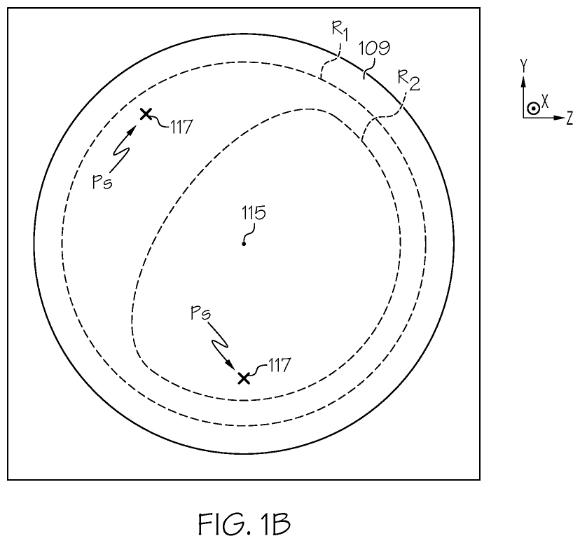

FIG. 1A shows an example of a known visual field test device 100. A screen 109 is positioned a distance D from a subject position 113 along the direction labelled X. The screen 109 has a uniform white or off-white color. The screen 109 in this example is substantially circular, as is best shown in FIG. 1B, and curves inwardly towards the subject position 113. A headrest 101 is provided at the subject position such that a patient may rest his head on the headrest 101 with his eye 111 facing the screen 109. In this example a focusing target 115 is present in the center of the screen 109, the purpose of which is to provide a point on the screen for the patient's eye 111 to focus on. The focusing target 115 could be provided by, for example, a mark applied to the screen 109 (e.g. a printed or painted mark) or by light directed from the screen towards the subject position 113 (e.g. a light-emitting diode (LED) disposed on the screen or light produced by the projector 107 and reflected towards the subject position 113).

A projector 107 projects light onto the screen 109 at a plurality of stimulus positions pₛ. The projector 107 may include, for example, one or more lasers, light-emitting diodes (LEDs), incandescent bulbs or other suitable means for generating light directed towards the screen 109. The light produced by the projector 107 is reflected by the screen 109 so as to produce visual stimuli 117 that are perceptible to the eye 111 of the patient at the subject position 113.

In a visual field test performed using the conventional visual field test device 100, the projector 107 is controlled so as to produce visual stimuli 117, one at a time, at different stimulus positions pₛ across the screen 109 while the patient's eye 111 is focused on the focusing target 115. The stimulus positions pₛ could be chosen in accordance with a predetermined pattern, for example a regular array of positions on the screen 109. The patient gives an indication each time he notices a stimulus 117, for example by pressing a button or by reporting to a technician supervising the visual field test, and the patient's feedback is analyzed to identify any positions on the screen 109 at which he failed to identify stimuli 117 when presented to him. This information can be used to measure the positions and extent of any defects or aberrations in the field of view of the patient.

FIG. 1B shows the screen 109 of the visual field test device 100 of FIG. 1A as viewed from the subject position 113. The focusing target 115 is in the center of the screen 109. Two stimuli 117 are shown at respective stimulus positions pₛ on the screen 109. As was explained above, the patient views the screen 109 with his eye 111 at the subject position 113 looking along the X direction and focused on the focusing target 115. The focusing target 115 lies in the center of the patient's field of view, and the stimuli 117 are offset (along the Y and Z directions) from this central position. Whether each stimulus 117 is perceptible to the patient during the visual field test thus depends on the extent of his peripheral vision.

The region labelled R₁ of FIG. 1B represents the field of view of a patient with no defects in his field of view having his vision focused on the focusing target 115. Both of the stimuli 117 shown in this Figure are within the region R₁, so the patient whose visual field it represents should be expected to correctly report seeing both stimuli 117 during a visual field test. The region labelled R₂ represents an example of the field of view of a patient whose field of view is truncated by a defect or aberration. Although one stimulus 117 appears within the region R₂, and should be identified by the patient during the visual field test, the other lies outside of it and as such the patient would be expected not to identify it during the visual field test. The failure of the patient to identify one of the stimuli would indicate that his field of view does not extend to its respective stimulus position pₛ.

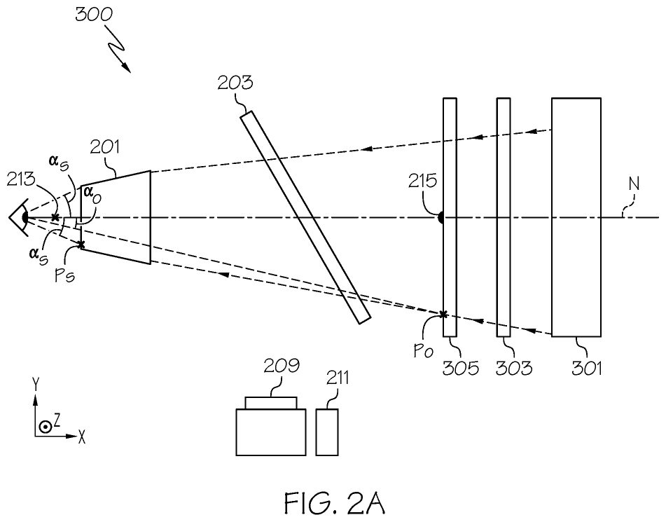

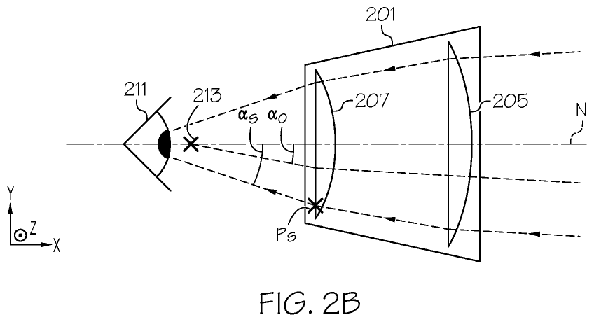

FIGS. 2A and 2B illustrate an embodiment of a visual field test device 200. The visual field test device 200 includes a light source 300, which comprises a backlight 301 that produces light directed towards a subject position 213. The light source 300 also includes a first liquid crystal display (LCD) screen 303 and a second LCD screen 305, each of which comprises an array of pixels individually controllable so as to vary the transmittance of the respective screen at each point in the respective array. In a visual field test conducted using the visual field test device 200, the first and second LCD screens 303, 305 may be controlled such that each screen presents a substantially uniform (e.g. dark) background, and then the transmittance of a small number of pixels in each screen may be changed (e.g. increased) so as to produce a relatively bright (or dark, if the background is configured to be comparatively bright) stimulus perceptible from the subject position 213. The light source 300 and its operation in a visual field test will be described in detail below.

Between the light source 300 and the subject position 213 is an eyepiece 201. The eyepiece 201 is configured to focus light received from the light source 300 towards the subject position 213 such that the light travels towards the subject position 213 at a steeper angle with respect to the line N between the subject position and the center of the light source, which in this case corresponds to the location of the focusing target 215, than the light would if it were to travel in a straight line from the light source 300 to the subject position 213. Hence, light output at a position p₀ on the light source 300 travels through the mirror 203 and the eyepiece 201 and, once output by the eyepiece 201, travels towards the subject position 213 at an angle αₛ. As a result, a patient positioned at the subject position 213 and looking towards the focusing target would perceive a stimulus 217 at a position pₛ that lies at a greater angle αₛ in his field of view (relative to the line N along which it is centered) than the angle α₀ of the direct line from the subject position 213 to the position p₀ on the light source 300 at which the light is output. (FIG. 2B shows an enlarged view of the subject position and angles described above.) The eyepiece 201 increases the range of angles in the field of view of the patient at which stimuli can be presented and enables the entire range of the peripheral vision of the patient to be studied.

As is shown in FIG. 2B, in this embodiment, the eyepiece 201 includes a first lens 205 and a second lens 207. Light from the light source 300 that is incident on the first lens 205 is focused towards the second lens 207, and focused again by the second lens 207 towards the subject position 213. In some embodiments, the eyepiece 201 may include any number of lenses (e.g., one or more, two or more, etc.) In some embodiments, eyepieces according to the present disclosure may include additional focusing structures such as mirrors. Embodiments of the present disclosure may include any eyepiece capable of focusing light towards the subject position at a sufficiently wide range of angles to present stimuli across the required range of the field of view of the patient could be implemented in this example.

Returning to FIG. 2A, in this embodiment the visual test device 200 includes, between the light source 300 and the subject position 213, a partial mirror 203, which permits the transmission of light output by the light source 300 (e.g., light produced by the backlight 301 and transmitted through the first and second LCD screens 303, 305) towards the subject position 213. The visual field test device 200 may also include an infra-red source 211 and a camera 209. The infra-red source 211 may be configured to produce infra-red radiation directed towards the mirror 203 that is thereby reflected towards the eyepiece 201 and onto the eye 211 at the subject position 213. The infra-red radiation may be reflected by the eye 211, through the eyepiece 201, and onto the mirror 203. The camera 209 may be positioned to record infra-red radiation reflected by the mirror 203 and analyze the received radiation to track the position of the pupil of the eye 211. By monitoring the position of the pupil it is possible to identify when the focus of the patient's vision deviations from the focusing target 215 and thus ensure that the patient's vision is focused in the appropriate direction throughout the test.

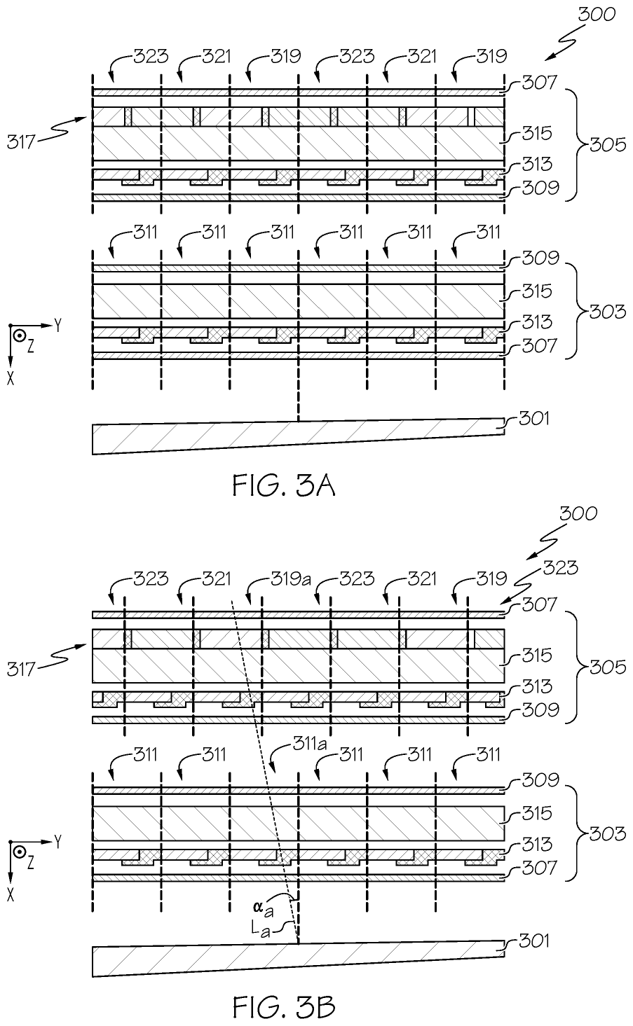

FIG. 3A illustrates a cross-sectional view of the light source 300. The backlight 301 is arranged behind (e.g., further along the X direction than) the first LCD screen 303 and the second LCD screen 305. The backlight 301 may incorporate any means of uniformly illuminating the first and second LCD screens 303, 305 with visible light, for example an array of LEDs, one or more fluorescent and/or incandescent bulbs or an electroluminescent screen. In some embodiments, the backlight 301 produces white light such that the first and/or second LCD screens 303, 305 can be controlled to filter some wavelengths in order to present differently-colored stimuli to the patient. In some embodiments the backlight may, for example, output a single color or a narrow range of colors.

The first LCD screen 303 may include a first polarizing filter 307, which permits the transmission of light polarized along the Y direction, and a second polarizing filter 309, which permits the transmission of light polarized along the Z direction (which is perpendicular to the Y direction). Between the first and second polarizing filters 307, 309 of the first LCD screen 303 may be a plurality of electrodes 313, which may be substantially transparent to visible light and may be arranged in a two-dimensional first array that extends in the Y and Z directions. Each electrode 313 may define respective pixel 311 in the first LCD screen 303.

Adjacent to the first array of electrodes 313 may be a liquid crystal layer 315. The liquid crystal layer 315 may be configured such that the liquid crystal layer 315 is capable of changing the direction of polarization of light transmitted through it in a manner that is dependent on the voltage applied to it. In a nematic liquid crystal, for example, in the absence of an electric field, the molecules of the liquid crystal align in a helical arrangement that can cause the polarization direction of light transmitted through the liquid crystal to rotate. When an electric field is applied, however, the molecules align with the electric field, reducing the strength of the helical ordering of the molecules and hence reducing the effect of the liquid crystal layer on the polarization direction of transmitted light. When the magnitude of the applied voltage is sufficiently large, the liquid crystal ceases to affect the polarization direction of the transmitted light. Light transmitted through a particular pixel 311 while a sufficiently large voltage is applied by the respective electrode 313 will thus be polarized along the Y direction by the first polarizing filter 307 and then, without experiencing any further polarization, encounter the second polarizing filter 309, which has a transmission direction (along the Z axis) which is perpendicular to that of the first polarizing filter 307 (along the Y axis). Since the light transmitted through the filter polarizing filter 307 is polarized perpendicular to the transmission direction of the second polarizing filter 309, no light will be transmitted through a pixel in which no voltage is applied. By varying the voltage applied by each electrode the 313, the transmittance of each respective pixel 311 can be hence be controlled. The first LCD screen may be colorless, i.e. does not absorb or scatter the light produced by the backlight 301 more strongly at some wavelengths than others. The first LCD screen 301 hence may modify the intensity of the transmitted light, and so, if subjected to white light from the light source 301, may have a greyscale appearance when viewed from the side of the second LCD screen 305.

Like the first LCD screen 303, the second LCD screen 305 may include a first polarizing filter 307, which permits the transmission of light polarized along the Y direction, and a second polarizing filter 309, which permits the transmission of light polarized along the Z direction. In the second LCD screen 305, however, the order of the first and second polarizing filters 307, 309 may be opposite to that of the polarizing filters in the first LCD screen 303 (that is to say that in the second LCD screen 305, the second polarizing filter 309 is further along the X direction that the first polarizing filter 307). The second LCD screen 305 may also include a plurality of electrodes 313 arranged in a second array (which is two-dimensional, extending throughout the second LCD screen 305 in the Y and Z directions) and a liquid crystal layer 315, which responds to a voltage as described above with reference to the liquid crystal layer 315 of the first LCD screen 303. Unlike the first LCD screen 303, the second LCD screen 305 may include a filtering layer 317. The filtering layer 317 may include an array of colored filters arranged in register (i.e. at equivalent positions in the plane of the Y and Z directions) with the second array of electrodes 313. Each colored filter may be either red, green or blue (to permit the transmission of one of red, green or blue light), and as a result the second array of pixels may include red pixels 319, green pixels 321 and blue pixels 323. By controlling groups of colored pixels 319, 321, 323 in the second LCD screen, the light source 300 can be made to produce stimuli of different colors as seen from the subject position 213.

It should be understood that, while in this example the first LCD screen 303, which is colorless, is positioned nearest the backlight 301, the first and second LCD screens 303, 305 could alternatively be arranged such that the second LCD screen 305 is nearer the backlight 301 (such that light output by the backlight 301 passes through the colored second LCD screen 305 before encountering the colorless first LCD screen 303). While in some embodiments one of the LCD screens 203, 205 is capable of coloring the light output by the light source 300, in other embodiments both the first and second LCD screens 303, 305 could be configured to be colorless.

In the embodiment of FIG. 3A, the pixels 311 in the first LCD screen 303 may be in register with the pixels 319, 321, 323 in the second LCD screen 305. This means that for each pixel 311 shown in the first LCD screen 303 there is a corresponding pixel in the second LCD screen 305 at the same position in the Y-Z plane (and extending across the same area of that plane). As a result, light can be permitted to pass through the first and second LCD screens 303, 305 along the direction of the X axis by controlling pairs of pixels at corresponding positions in the Y-Z plane. This arrangement may be preferable where the eyepiece 201 of the visual field test device is configured to receive parallel rays of light from the light source 300 and direct them towards the subject position 213. In some embodiments, however, the eyepiece 201 is adapted to receive non-parallel rays of light from the light source 300. For example, the eyepiece 201 may be configured to receive rays of light that are angled so as to converge as they travel along the X axis towards the eyepiece 201.

FIG. 3B shows an embodiment of a light source in which the arrays of pixels in the first and second LCD screens 303, 305 are offset with respect to one another along the Y axis such that each pixel 311 in the first LCD screen 303 is approximately halfway between two respective pixels in the second LCD screen 305. Thus, if the pixel labelled 311a in the first LCD screen 303 and the red pixel labelled 319a in the second LCD screen 305 are both controlled so as to increase their respective transmittances relative to the surrounding pixels, a comparatively red spot may be displayed on the light source 300 that is brightest when viewed along the direction of the line Lₐ, which is at an angle αₐ to the X axis.

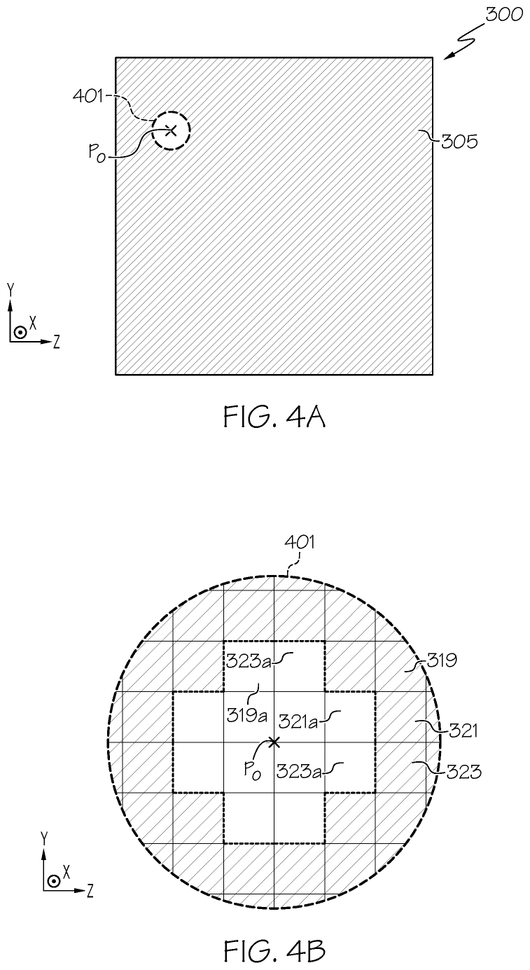

FIGS. 4A and 4B illustrate the light source 300 as viewed along the X axis from the side of the subject positon 213. The second LCD screen 305 is visible when viewed from this side, and the appearance of the light source 300 depends on how both the first and second LCD screens 303, 305 are controlled so as to permit the transmission of light from the backlight 301 towards the subject position 213. In this example, the first and second LCD screens 303, 305 are both controlled such that the transmittance of each screen is a low, uniform value (e.g. 1 per cent) everywhere except for within a region 401 centered on an output position p₀. In the region 401, groups of pixels at corresponding positions in each of the first and second LCD screens 303, 305 have been set at a value substantially higher than that of the uniform background (e.g. 95 per cent), and which produces a perceptible bright spot in the region 401.

An enlarged view of the region 401 is shown in FIG. 4B. It can be seen that the group of pixels 319a, 321a, 323a appear substantially brighter than the surrounding pixels 319, 321, 323 as a result of their increased transmittances.

As a result of the combination of two LCD screens 303, 305, it is possible to achieve a large contrast between the region 401 of the bright spot and the background. If the transmittance of each screen in the chosen background region is about 1 per cent of the maximum transmittance of the screen, then the combined transmittance of the first and second LCD screens 303, 305 across the background region will be approximately 0.0001 of the maximum combined transmittance, and if the transmittance of a group of pixels in the region 401 of the bright spot is on the order of 1, the intensity of the bright spot will on the order of 10,000 times greater than that of the background. The light source 300 can thus achieve the high values of dynamic range required to perform visual field tests while allowing the visual field test device 200 to be made compact relative to known devices (such as that illustrated in FIGS. 1A and 1B), since the combination of the eyepiece 201 with this light source 300 enables stimuli to be presented across a sufficiently large range of angles within the field of view of a patient at the subject position 213 to perform a complete visual field test.

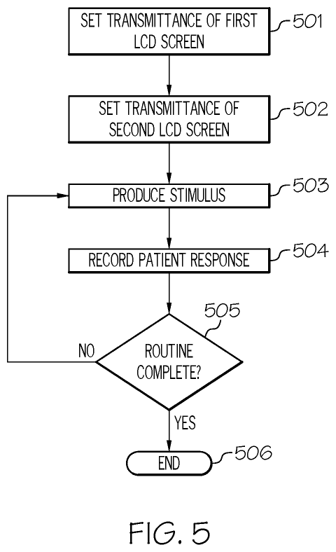

FIG. 5 shows a flowchart for an embodiment of a method of performing a visual field test. This method could be implemented using the visual field test device of FIGS. 2A and 2B, for example, and will be described with reference to the apparatus discussed above.

At step 501 the pixels 311 of the first LCD screen 303 are set to each have a uniform, relatively low transmittance. The transmittance of each pixel 311 in the first LCD screen 303 could be set to 1 per cent, for example. At step 502, the pixels 319, 321, 323 of the second LCD screen 305 are also controlled so as to each have a low and uniform transmittance (e.g. one per cent, or some other value). As a result of steps 501 and 502, the light source 300 appears, when viewed from the subject position, presents to the patient a uniform background (which could be dark). It should be understood that steps 501 and 502 could be performed in any order or simultaneously.

At step 503, a pixel or group of pixels at corresponding positions in each of the first and second LCD screens 303, 305 may be controlled so as to increase the transmittance of those pixels relative to the uniform background produced by steps 501 and 502. The light output by the light source at the positions of these pixels appears as a bright spot at an output position p₀ on the light source 300 against the comparatively dark background when viewed along the X axis from the side of the subject position 213. As was explained above with reference to FIGS. 3A and 3B, the groups of pixels controlled in each screen to produce the stimulus may be chosen to maximize the brightness of the bright spot along the direction from which the eyepiece 201 is configured to receive light from the light source 300. This could be the X direction or a direction oblique to it (as shown in FIG. 3B). The second LCD screen 305 could be controlled such that the bright spot is colored (by permitting the transmission only through a combination of pixels 319, 321, 323 corresponding to the desired color) or white (by permitting the transmission of light through equal numbers of red, green and blue pixels 319, 321, 323).

Light from the produced bright spot is received by the eyepiece 201 and directed towards the subject position 213, leading to the appearance of a stimulus 217 at a respective stimulus position pₛ in the field of view of a patient at the subject position 213. At step 504, the response of the patient while presented with the stimulus is recorded. This step could include, for example, recording whether the patient consciously indicates that he has seen the presented stimulus (e.g. by pressing a button or by communicating with a technician) and/or monitoring the pupil of the patient's eye 211 using the camera 209.

The visual field test may be defined by a routine that involves presenting several stimuli, e.g. of different colors and at different positions, to the patient. At step 505, if the routine has not yet been completed, the next stimulus is presented to the patient as described above and his response to it is again recorded. If the routine is complete, the test proceeds to step 506 and ends.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A visual field test device comprising:
a light source comprising a backlight arranged to generate, in use, light directed towards a first liquid crystal display (LCD) screen and a second LCD screen, the first LCD screen and the second LCD screen overlapping one another and the backlight such that the light source outputs light transmitted through both the first and second LCD screens; and
an eyepiece arranged between the light source and a subject position, the eyepiece being configured to receive light output by the light source and focus the received light towards the subject position;
wherein each of the first and second LCD screens comprises a respective array of pixels, each pixel being controllable so as to vary, in use, its transmittance to light generated by the back-light such that changing the transmittance of one or more pixels at corresponding positions in each of the first and second LCD screens relative to the surrounding pixels produces a stimulus perceptible when viewed through the eyepiece from the subject position.

2. The visual field test device of claim 1, wherein the eyepiece is configured to focus the received light towards the subject position across a range of angles having a magnitude greater than the angle subtended by the light source when viewed from a distance equal to the distance between the subject position and the light source.

3. The visual field test device of claim 1, wherein the pixels of the first LCD screen are colorless.

4. The visual field test device of claim 1, wherein the array of pixels of the second LCD screen comprises pixels each having one of a plurality of colors, the plurality of colors comprising red, green and blue.

5. The visual field test device of claim 1, wherein the first LCD screen is arranged between the back-light and the second LCD screen.

6. The visual field test device of claim 1, further comprising a focusing target positioned between the back-light and the subject position such that a patient at the subject position is able to focus his gaze on the focusing target.

7. The visual field test device of claim 1, wherein the separation between the eyepiece and the subject position is less than the separation between the eyepiece and the light source.

8. The visual field test device of claim 1, further comprising a feedback device configured to receive feedback from a patient at the subject position during a visual field test in which one or more stimuli perceptible at the subject positon are produced by the light source.

9. The visual field test device of claim 1, further comprising a camera configured to monitor the position of a pupil of the eye of a patient at the subject position.

10. The visual field test device of claim 9, further comprising an illuminating source configured to illuminate the subject position with radiation detectable by the camera such that such that the camera monitors the position of the pupil of the eye by recording the radiation reflected by the eye.

11. The visual field test device of claim 10, wherein the radiation comprises one or more infra-red wavelengths.

12. The visual field test device of claim 8, further comprising an optical component arranged to direct the radiation produced by the illuminating source towards the eye and/or direct the radiation reflected by the eye towards the camera.

13. The visual field test device of claim 8, wherein the illuminating source is configured to direct the radiation towards the eyepiece, whereby the radiation is directed towards the subject position.

14. The visual field test of claim 13, wherein the optical component comprises a partial mirror arranged between the light source and the eyepiece, the partial mirror being configured to:
permit light from the light source to be transmitted therethrough towards the eyepiece, and
to reflect the radiation produced by the illuminating source towards the eyepiece, whereby the radiation is directed towards the subject position, and/or reflect the radiation reflected by the eye towards the camera.

15. A method of performing a visual field test, the method comprising:
controlling a backlight to generate light directed towards a first LCD screen and a second LCD screen, the first LCD screen and the second LCD screen overlapping one another and the backlight such that the light source outputs light transmitted through both the first and second LCD screens, wherein each of the first and second LCD screens comprises a respective array of pixels, each pixel being controllable so as to vary, in use, its transmittance to light from the back-light, and wherein an eyepiece is arranged between the light source and a subject position, the eyepiece being configured to receive light output by the light source and focus the received light towards the subject position; and
changing the transmittance of one or more pixels at corresponding positions in each LCD screen relative to the surrounding pixels so as to produce a stimulus perceptible when viewed through the eyepiece from the subject position.

16. The method of claim 15, wherein the pixels of each of the first and second LCD screens outside of a first region are controlled so as to produce a uniform background which surrounds the first region and in which the luminance of the light source as seen from the subject position is less than that of the first region.

17. The method of claim 16, wherein the pixels of the first and second LCD screens are controlled such that the contrast ratio between the luminance of the first region and that of the uniform background as perceived from the subject position is at least 10,000:1.

18. The method of claim 15, further comprising controlling the first and second LCD screens to as to produce a focusing target perceptible from the subject position and suitable for focusing the vision of a patient at the subject position.

19. The method of claim 15, further comprising presenting a plurality of stimuli each at a respective position on the light source, each respective position corresponding to a position in the field of view of a patient at the subject position.

20. The method of claim 19, wherein the plurality of stimuli are presented in accordance with a predetermined routine.
